(19) United States
(12) Patent Application Publication
(10) Pub. No.: US 2016/0228584 A1
(43) Pub. Date: Aug. 11, 2016

(54) 18F -GLUTATHIONE CONJUGATE AS A PET TRACER FOR IMAGING TUMORS OR NEUROLOGICAL DISORDERS THAT OVEREXPRESS L-PGDS ENZYME

(71) Applicant: National Tsing Hua University, Hsinchu (TW)

(72) Inventors: Kun-Ju LIN, Hsinchu (TW); Chun-Nan YEH, Hsinchu (TW); Ying-Cheng HUANG, Hsinchu (TW); Ho-Lien HUANG, Hsinchu (TW); Chung-Shan YU, Hsinchu (TW)

(21) Appl. No.: 14/957,081

(22) Filed: Dec. 2, 2015

(30) Foreign Application Priority Data
Feb. 11, 2015 (TW) 104104543

(51) Int. Cl. A61K 51/04

(52) U.S. Cl. CPC A61K 51/0402

(57) ABSTRACT

A PET tracer for imaging tumors or neurological disorders that overexpress L-PGDS enzyme is provided. The present invention have prepared a glutathione conjugate of fluorine-18-labeled fluorobutyl ethacrynic amide using an acceptable amount of radioactivity that is capable of binding to L-PGDS and can be used for in vitro and in vivo imaging studies.

### BACKGROUND OF THE INVENTION

1. Field of the Invention

The present invention relates to a PET tracer, particularly to ¹⁸F-glutathione conjugate as a PET tracer for imaging tumors or neurological disorders that overexpress L-PGDS enzyme.

2. Description of the Prior Art

Positron emission tomography (PET) has become a functional imaging technique in medical diagnostics. Molecules (e.g. drugs) and biological macromolecules (e.g. proteins) in vivo imaging rely on the positron emitters (e.g. fluoro-18). The characteristics of ¹⁸F, such as low radiation doses, short tissue range, feasibility of multi-step synthesis and extendable scanning protocols are attributed to a relatively low energy of 0.64 MeV and a relatively long half-life (t_{1/2}=109.7 min).

The adequate atomic size due to being a member of the second periodic atoms makes ¹⁸F a suitable atom for mimicking oxygen or hydrogen. The high sensitivity of ¹⁸F allows the use of a very low concentration (10⁻¹² M) of radio-labeled tracer for imaging cellular markers, for example, receptors without encountering toxicity concerns.

Introduction of ¹⁸F can be mediated through a direct substitution reaction or an indirect reaction via a bifunctional group. The former includes a nucleophilic or electrophilic pathway. The bifunctional group is also named prosthetic group or synthon of ¹⁸F.

Thus, it needs further development and application of ¹⁸F tracer for PET imaging.

### SUMMARY OF THE INVENTION

The present invention is directed to developing ¹⁸F-glutathione conjugate as a PET tracer for imaging tumors or neurological disorders that overexpress L-PGDS enzyme.

According to one embodiment of the present invention, a PET (positron emission tomography) tracer is used for imaging a tumor or a neurological disorder that overexpresses L-PGDS (Lipocalin-type prostaglandin D synthase) enzyme. The PET tracer comprises formula (3) or pharmaceutical salts thereof.

According to another embodiment of the present invention, a method for imaging a tumor or a neurological disorder that overexpresses L-PGDS (Lipocalin-type prostaglandin D synthase) enzyme comprises administering an effective amount of the above-mentioned PET tracer to a subject; and performing a PET imaging to the subject so as to imaging the tumor or the neurological disorder that overexpresses L-PGDS enzyme.

Other advantages of the present invention will become apparent from the following descriptions taken in conjunction with the accompanying drawings wherein certain embodiments of the present invention are set forth by way of illustration and examples.

### BRIEF DESCRIPTION OF THE DRAWINGS

The foregoing aspects and many of the accompanying advantages of this invention will become more readily appreciated as the same becomes better understood by reference to the following detailed descriptions, when taken in conjunction with the accompanying drawings.

FIGS. 1a to 1c illustrate purification using chiral analytic RP-HPLC and semipreparative RP-HPLC. FIG. 1a illustrates that the racemic mixture of nonradioactive FBuEA-GS 3 was resolved to two components by chiral analytic RP-HPLC. The major peak A and the minor peak B represent the presence of two isomers. Injection volume: 0.01 mL from the sample with concentration of 1 mg/0.2 mL. FIG. 1b illustrates typical chromatogram of [¹⁸F]FBuEA-GS 3 after purification with semipreparative RP-HPLC. Injection volume: 0.01 mL from the purified sample with concentration of 440 μCi/0.2 mL. FIG. 1c illustrate the HPLC chromatogram of the purified [¹⁸F]FBuEA-GS 3 co-mixed with the authentic sample using semipreparative RP-HPLC. Injection volume: 0.2 mL from authentic sample with concentration of 0.02 mg/0.2 mL.

FIG. 2 illustrates inhibition of the formation of PGD₂ from PGH₂ in the presence of 200 μM of each test compound.

FIG. 3 illustrates Ex vivo analysis of the distribution of [¹⁸F]FBuEA-GS 3 in a rat.

FIGS. 4a to 4b illustrate images of PET and MRI of brain of a C6-glioma rat. FIG. 4a illustrates dynamic PET images taken over 20-40 min at three cross sections. Lower right shows an MRI image. FIG. 4b illustrates Images of the coronal cross section at different time frames (10-30 min, 30-60 min, 60-90 min and 90-120 min) post injection of [¹⁸F]FBuEA-GS 3. PET scanner description: microPET R4; Concorde Microsystems Inc. Injection dose: 1.58 mCi/0.5 mL.

### DESCRIPTION OF THE PREFERRED EMBODIMENT

Lipocalin type prostaglandin D synthase (L-PGDS) is expressed in the brain and is reportedly implicated in neurological disorders such as Alzheimer's disease and tumors such as brain tumor, breast cancer or ovarian cancer. In addition, L-PGDS constitutes one of the most abundant proteins in the cerebrospinal fluid.

The present invention have prepared a glutathione conjugate of fluorine-18-labeled fluorobutyl ethacrynic amide or pharmaceutical salts thereof that is capable of binding to L-PGDS and can be used for in vitro and in vivo imaging studies.

In one embodiment, the glutathione conjugate of fluorine-18-labeled fluorobutyl ethacrynic amide is represented by formula (3) and abbreviated as [¹⁸F]FBuEA-GS 3, wherein [¹⁸F]FBuEA-GS 3 may be chiral or mixtures thereof.

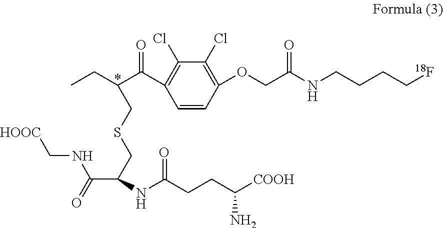 [SMILES: CCC(CSC[C@@H](NC(=O)CC[C@@H](N)C(=O)O)C(=O)NCC(=O)O)C(=O)c1ccc(OCC(=O)NCCCC[18F])c(Cl)c1Cl]

As used herein, the term “pharmaceutically acceptable salts” refers to salts or zwitterionic forms of the compounds disclosed herein. Salts of such compounds can be prepared during the final isolation and purification of the compounds or separately by reacting the compound with an acid having a suitable cation. Suitable pharmaceutically acceptable cations include alkali metal (e.g., sodium or potassium) and alkaline earth metal (e.g., calcium or magnesium) cations. In addition, the pharmaceutically acceptable salts of the disclosed compounds that contain a basic center are acid addition salts formed with pharmaceutically acceptable acids.

Examples of acids which can be employed to form pharmaceutically acceptable salts include inorganic acids such as hydrochloric, hydrobromic, sulfuric, and phosphoric, and organic acids such as oxalic, maleic, succinic, malonic, lactic and citric. In light of the foregoing, any reference to compounds appearing herein is intended to include compounds disclosed herein as well as pharmaceutically acceptable salts, solvates (e.g., hydrates), esters, or prodrugs thereof.

The preparation and application of the present invention may be further detailed in PLoS One 2014 11; 9(8):e104118. Epub 2014 Aug. 11, herein incorporated by reference in its entirety.

In one embodiment, a method for imaging a tumor or a neurological disorder that overexpresses L-PGDS (Lipocalin-type prostaglandin D synthase) enzyme, comprising administering an effective amount of a PET tracer comprising formula 3 or pharmaceutical salts thereof to a subject; and performing a PET imaging to the subject so as to imaging the tumor or the neurological disorder that overexpresses L-PGDS enzyme.

The present invention is further illustrated by the following working examples in combination of accompany figures, which should not be construed as further limiting.

General

[¹⁸F]HF was produced with a PET tracer cyclotron (GE, TR-30) via the ¹⁸O(p,n)¹⁸F nuclear reaction at Nuclear Energy Research institute (NERI), Taiwan. The radiochemical experiment was performed with a GE Tracer LAB FXFN synthesis module (GE medical systems, Milwaukee, Wis.). The crude mixture[¹⁸F]FbuEA 2 in TracerLAB FXFN synthesis module was purified using reversed phase high performance liquid chromatography (RP-HPLC), consisting of a Waters 510 pump and a linear UVIS detector (Δ=254 nm) in series with a Bertholdc-flow detector (Raytest, GABI Star) and a RP-18 column CHEMCOSORB 7-ODS-H, 10×250 mm, 5 μm. The identity of the labeled compound [¹⁸F]FbuEA 2 was confirmed by comparing with the authentic compound on HPLC chromatogram. The UV absorbance peak at 254 nm was integrated for comparing with the standard curve relating mass to UV absorbance. Only a specific activity below 40 GBq/μmol can be measured accurately. Radioactivity was measured with a Capintec R15C dose calibrator. Recombinant human glutathione S-transferase alpha-1 (GSTA1 human, 50 μg/50 μL) was purchased from Pro SpecTany Techno Gene Ltd (ENZ-469). Recombinant human glutathione S-transferase Pi-1 (GSTP1 human, 25 μg/25 μL) were purchased from Alpha Diagnostic International Inc. (GST P35-R-25). The enzymes of L-PGDS and m-PGES 1 were purchased from Cayman Chemical Inc. All these enzyme products were freshly unpacked and immediately used for enzymatic assay. HPLC system used for binding assay included a Waters 510 pump and a linear UVIS detector (λ=254 nm) that was assembled in series with a Berthholdc-flow detector on a TSKgel G3000 PW 7.5×300 (mm) with a particle size of 10 μm, which was purchased from Tosoh Bioscience LLC. Flow rate was setting at 1 mL/min.

PET imaging was performed with microPET R4 (Concorde Microsystems Inc.) and a NanoPET/CT (MEDISO Inc.) in Nuclear Energy Research Institute. Both the machines were manufactured by Siemens Medical Solutions, Knoxville, United States.

Radiochemical synthesis of [¹⁸F]FBuEA-GS 3

Preparation of compound 3 is proposed as follows.

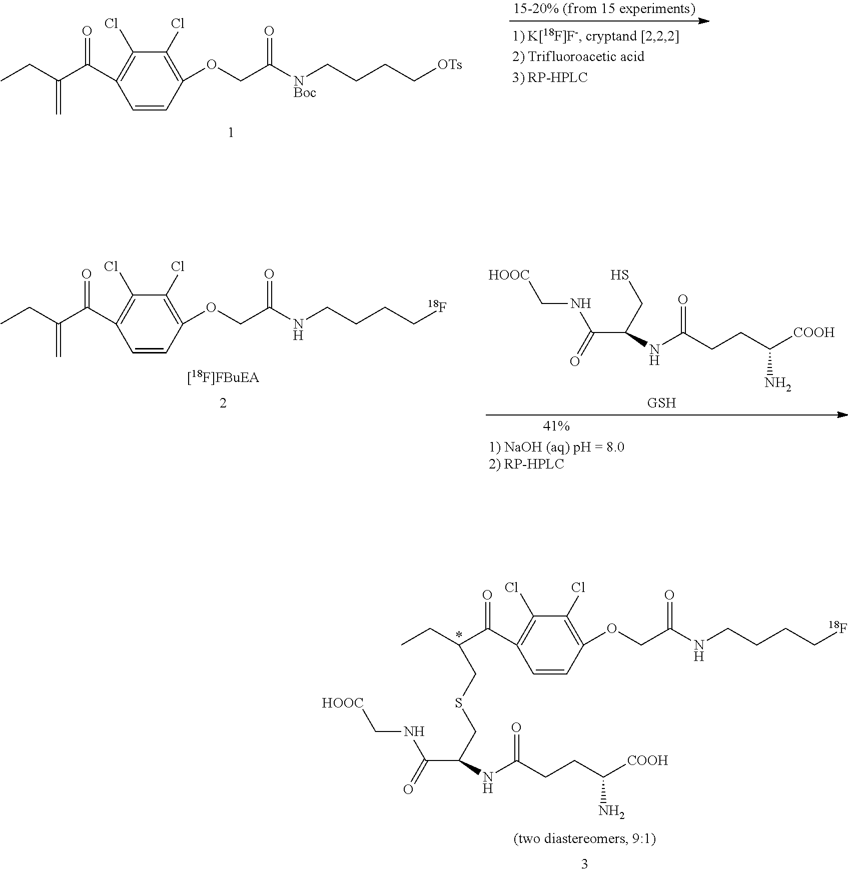 [SMILES: C=C(CC)C(=O)c1ccc(OCC(=O)N(CCCCOS(=O)(=O)c2ccc(C)cc2)C(=O)OC(C)(C)C)c(Cl)c1Cl ; C=C(CC)C(=O)c1ccc(OCC(=O)NCCCC[18F])c(Cl)c1Cl ; N[C@H](CCC(=O)N[C@H](CS)C(=O)NCC(=O)O)C(=O)O ; CCC(CSC[C@@H](NC(=O)CC[C@@H](N)C(=O)O)C(=O)NCC(=O)O)C(=O)c1ccc(OCC(=O)NCCCC[18F])c(Cl)c1Cl]

In brief, [¹⁸F]FBuEA 2 was prepared from [¹⁸F]F⁻ (824 mCi) with the tosylate 1 through purification with a series of cartridge settings in a synthetic module. The fluorination agent was obtained from 3.5 mg K₂CO₃, 0.5 mL H₂O and crypt and [2,2,2] (15 mg)/CH₃CN (1 mL). In addition, t-BuOH (0.4 mL) was used during the fluorination procedure.

The mixture of compound 2 was further purified using HPLC settings as described above. The flow rate was 3 mL/min. The gradient settings starts from 20% CH₃CN (aq.) obtained by mixing CH₃CN and 0.05% trifluoro acetic acid, via 95% CH₃CN solution at 10 min, to a final 100% CH₃CN solvent at 20 min. t_{R}=14.8 min. The preparation along with purification with semipreparative RP-HPLC was accomplished within 1 h. A portion (7 mCi, 0.2 mL) drawn off from the collected fractions (3 mL, 82 mCi) was transferred to a round-bottomed flask (10 mL) followed by concentration under reduced pressure using membrane pump to obtain the residue. To the residue was added CH₃CN (1 mL), H₂O (1 mL), and GSH (20 mg), sequentially. A solution of aqueous NaOH (50 mM) was added until the pH was adjusted to 8.2 (0.6 mL, within 1 min). The stirring was allowed for 15 min. After filtration with 0.45 mM Nylon filter (Merck), the filtrate (2.6 mL) was purified using semipreparative RP-HPLC. The column setting was the same as that described for compound 2. The gradient settings were the same as that described above Retention time (t_{R}) of [¹⁸F]FBuEAGS 3 was 14.6 min. The fractions collected (6 mL) were concentrated under reduced pressure using membrane pump for 10 min to provide [¹⁸F]FBuEA-GS 3 in 5% radiochemical yield (2.05 mCi) with specific activity of 33 GBq/μmol and radiochemical purity of 98%, based on the calculation of initial radiofluoride ion [¹⁸F]F⁻ (824 mCi).

For each group of experiment, a volume of 0.01 mL was drawn from a concentration of 440 μCi/0.2 mL of the purified [¹⁸F]FBuEA-GS 3. Synthesis and purification of [¹⁸F]FBuEA-GS 3 from [¹⁸F]FBuEA 2 was completed within 1 h. The whole preparation along with purification with semipreparative RP-HPLC starting from radiofluoride ¹⁸F⁻ was completed in 2 h. Nonradioactive FBuEA-GS 3 was prepared separately and analyzed by RP-HPLC as described above except that an analytic chiral column (Chiralcel OD-RH 0.46×15 cm, Daicel Chemical Industries, LTD.) was used instead. The gradient setting was the same as above described and the flow rate was 0.7 mL/min.

Bioassay of Competitive Inhibition of FBuEA-GS 3 Against the Production of PGD₂ from PGH₂

This assay was performed according to the protocol described by the commercial kit (Cayman cat. No. 10006595). In brief, this method was divided to two parts. Part one was regarding the assay of production of PGD₂ from PGH₂ under the catalysis of L-PGDS. The formation of PGD₂ could be inhibited by AT-56, a dibenzocycloheptenyl tetrazolyl piperidine. To compare with the inhibition by FBuEA-GS 3, uridine was employed as a negative control. Part two was regarding the determination of the concentration of PGD₂ by enzyme immune assay (EIA) of the PGD₂-conjugate as a competitor. The conjugate linked by acetylcholine esterase and PGD₂ binds competitively to an immobilized antibody. After wash, the residual conjugate could catalyze the hydrolysis of acetylcholine and the released thiocholine replaces one thio group of 5,59-dithio-bis-2-nitrobenzoic acid yielding a colored 5-thio-2-nitrobenzoic acid with absorbance of UV at λₘₐₓ of 412 nm. The intensity of absorbance is inversely proportional to the concentration of PGD₂ derived from PGH₂. Thus, the more intensive absorbance the detector senses, the more effective inhibition the substrate exerts. Before performing the assay, a calibration curve by plotting the activity detected vs. concentration of PGD₂ as the competitor was constructed. Throughout the whole assay for the three substrates, the percentages of activities ranging from 41.4%-61.2% were lying in a reliable linear detection between 26.8% (15000 pg/mL) and 76.9% (468.8 pg/mL). The inhibition percentage was calculated as [(Absᵢₙᵢₜᵢₐₗ−Abs_{control})−(Abs_{inhibitor}−Abs_{control})]/(Absᵢₙᵢₜᵢₐₗ−Abs_{control})×100%. Experiments were performed in duplicate.

Assay of Binding of Radioligand to Enzymes Tested

The aforementioned [¹⁸F]FBuEA-GS 3 was diluted with distilled H₂O (1 mL). An aliquot (20 mL) was drawn off to each of the eppendorfs of the enzyme solution. The whole mixture was incubated at 25 μC for 15 min followed by analysis using HPLC coupled with gel filtration column (TSKgel G3000PW 7.5×300, 10 mm, Tosoh Bioscience LLC). Distilled H₂O was employed as the eluent. The flow rate was 1 mL/min.

Determination of Binding Constant (K_{d}) of [¹⁸F]FBuEA-GS 3 to L-PGDS

An amount of 250 mg/200 mL of the commercial L-PGDS (human recombinant, Cayman, No. 10006788) was mixed with tris-HCl buffer (50 μL, 100 μM, pH=8.0) to provide the stock solution (250 μg/250 μL). An aliquot (10 μL) drawn from the stock was added to an eppendorf (200 μL). A solution of [¹⁸F]FBuEA-GS 3 in tris-HCl buffer solution (0.40 mCi/5 mL) was added. A carrier solution of nonradioactive FBuEA-GS 3 was prepared via a series of dilution from a stock to provide various samples in concentration of 4, 30, 80, 600, 1600 and 4800 μM. A volume of 5 mL for each sample was added to the above eppendorf to generate the final concentration of 1, 7.5, 20, 150, 400 and 1200 μM. As a control, 5 μL of tris-HCl solution was used. The mixture was immediately (5 sec.) transferred to HPLC for binding analysis. The other assay group using HPLC for equilibrium of 10 minutes followed the same condition except that the equilibrium time was extended to 10 min.

Study of the Cellular Uptake of [¹⁸F]FBuEA-GS 3

The freshly prepared [¹⁸F]FBuEA-GS 3 was diluted with medium (DMEM, 5% FBS) to a concentration of 10 mCi/50 mL in a centrifuge tube. When the cells were grown in microtiterplates for 24 h, the growth medium (500 mL) was replaced with a mixture of [¹⁸F]FBuEA-GS 3 in 500 mL followed by incubation at 37 μC. The time point of addition of radio tracer was staggered such that every group could be harvested concurrently. At various times of 0.25, 0.5, 1.5, 3 and 5 h, the collection of the medium was progressing. During harvesting, the radioactive medium was collected from each of the wells, followed by rinsing with PBS 500 mL twice. The medium and rinses (1.5 mL) were combined for counting; the counts were treated as extracellular radioactivity. Subsequently, the cells were lysed with 0.25% trypsin-EDTA (30 mL) and the wells were rinsed with PBS twice. Both cells and rinses (1.5 mL) were combined for counting; the counts were treated as intracellular radioactivity. Radioactivity was measured using a scintillation gamma counter (Packard 5000, Packard Instrument Co. laboratory) and decay was corrected. Samples were performed triplicated at each time point for all uptake studies.

The uptake ratio was calculated according to the following expression:

Uptake ratio(%)=Count_{intracellular}=Count_{extracellular}+Count_{intracellular})×100%

Rat Model

All in vivo experiments were performed in compliance with the NHMRC Taiwan Code of Practice for the care and use of animals for scientific purposes. Affidavit of approval of Animal Use Protocol Chang Gung Memorial Hospital, No 2013092702 and CGU12-055 was granted before performing the assessment. Sprague-Dawley (SD) rats (8 weeks of age) were obtained from the BioLasco animal Co. (Taiwan). Rats were housed under constant environmental conditions and were allowed free access to food and water throughout the experimental period. The rats were anaesthetized via inhalant isoflurane (Forthane, Abott) in 200 mL/min oxygen during the imaging study.

All studies involving animals were conducted in compliance with federal and institutional guidelines. Two weeks before imaging, healthy male SD rats were stereotactically inoculated in the right hemisphere with 1.0×10⁵ C6 glioma cells (American Type Tissue Collection).

After C6 glioma cells were injected into the striatum of the SD rats, the animals were placed on heating pad until they have entirely recovered. When the xenografted tumor size has grown to a size of 1-2 mm in diameter, the animals were transferred to the animal facility under control by the research staff every morning. The animals were visited at least daily for signs of pain or distress; If the animals appear lethargic, do not appear to be eating or drinking over 24 hours, or weight loss greater than 20% body weight, euthanasia will be carried out to avoid further suffering. Prior to imaging, all rats were affixed with venous and arterial catheters. The in vivo xenograft C6 glioma was imaged 2 weeks after transplantation procedure, until the volume of the tumor reached 3 mm-5 mm in diameter, well-demarcated from normal brain tissue. Regular oral feeding was proceeded after the animals were recovered from anesthesia.

The animals were monitored regularly with care in respect to the feeding quality, interaction, and symptom of dystrophy. The animal care unit controlled the abnormalities such as the feeding intake ratio less than 50% in 72 hours, hind leg paraparesis or the weight loss greater than 20%. As long as one of the above conditions was met, the animal will be sedated with the ketamine and xylazine hydrochloric acid combination followed by euthanasia with CO₂ with xylocaine (200 mg) intravenously.

Ex Vivo Analysis of the Biodistribution of [¹⁸F]FBuEA-GS 3

Fourteen specimens were isolated after the injection of [¹⁸F]FBuEA-GS 3 in activity ranging from 0.9 to 1.2 mCi. The five rats were each used for provision of the specimens at the five time points of 15, 30, 60, 90 and 120 minutes post injection of [¹⁸F]FBuEA-GS 3. The specimens included 1) organ tissues such as brain, liver, spleen, heart, kidney, lung, colon, small bowel, stomach, testes, skull, and muscle, and 2) body fluids such as blood and urine. These specimens were submitted for counting the radioactivity using a solid scintillation gamma counter (Packard 5000, Packard Instrument Co. laboratory). The counting value of each specimen was further divided by the sample weight to give the final expression as percentage of injection dose per sample weight (% ID/g).

HPLC Radiometabolite Analysis

An amount of 2.14-2.72 mCi of [¹⁸F]FBuEA-GS 3 obtained as described above was dissolved in saline solution (0.2-0.3 mL). The injection dose for each of the 5 rats was in the range from 0.9 to 1.14 mCi per 0.1 mL except the group for 60 min experiment that used 0.3 mL. Arterial blood (2 mL) was collected at 15, 30, 60, 90, and 120 min from each of the 5 rats. After centrifugation with 3500 rpm at rt for 5 min, the supernatant (0.5 mL) was then mixed with the nonradioactive authentic FBuEA-GS (10 mL drawn from 1 mg/1 mL) followed by semipreparative RP-HPLC investigation using the gradient condition as described above. Radiochromatographic data were recorded and collected using a radioisotope detector (Bioscan, Washington, D.C., USA).

Immunohistological Stainings

The whole brains of a rat were harvested and fixed in 4% formalin for 48 hours followed by paraffin embedding for immunohistological stainings. Tissue sections were detached in thickness of 5.0-mm followed by staining with the kit of L-PGDS specific rabbit polyclonal antibody (Novus, NBP1-79280). Immunoactive spots were assessed using a horseradish peroxidase detection kit (Dako, Glostrup, DK). Hematoxylin and eosin staining was used to evaluate the cell density and tumor localization.

MRI Imaging

MRI was used to localize the site of C6 tumor lesions. Rats were secured prone in a radiofrequency coil (38-mm inner diameter) and placed in a 4.7-T horizontal bore imaging system (Varian Inc., Palo Alto, Calif., USA). A constant body temperature at 37 uC was maintained using heated airflow. An initial multislice gradient echo imaging sequence (repetition time, 150 ms; echo time, 3.5 ms; matrix, 128×128; field of view, 40×40 mm²; slice thickness, 2 mm) was used to acquire 7 slices for each of axial, coronal and sagittal imaging plane for proper positioning of subsequent scans.

A multislice T2-weighted fast spin-echo scan with 8 echoes and 8.0-ms echo spacing (effective echo time, 32 ms) was then collected using the parameters of a repetition time of 2,000 ms, field of view of 32×32 mm², matrix of 128×128, 16 acquisitions and 8 coronal slices in thickness of 2-mm.

PET/CT Imaging

PET scanning experiments were performed within 72 hours of MRI experiment that used to confirm a successful inoculation of tumors by administering [¹⁸F] FBuEA-GS3 via tail vein injection. Both machines of microPET and nanoPET/CT were employed.

Data were collected in list-mode format for 120 minutes. For reconstruction, the dynamic PET acquisition was divided into six 20-min frames over the scanning duration.

The raw data within each frame were then binned into three-dimensional sinograms, with a span of three and ring difference of 47. The data were corrected for scattering and attenuation using a two-dimensional ordered-subsets expectation-maximization algorithm with 16 subsets and four iterations. The sonograms were reconstructed into tomographic images (128×128×95) with voxel sizes of 0.095×0.095×0.08 cm³.

Results

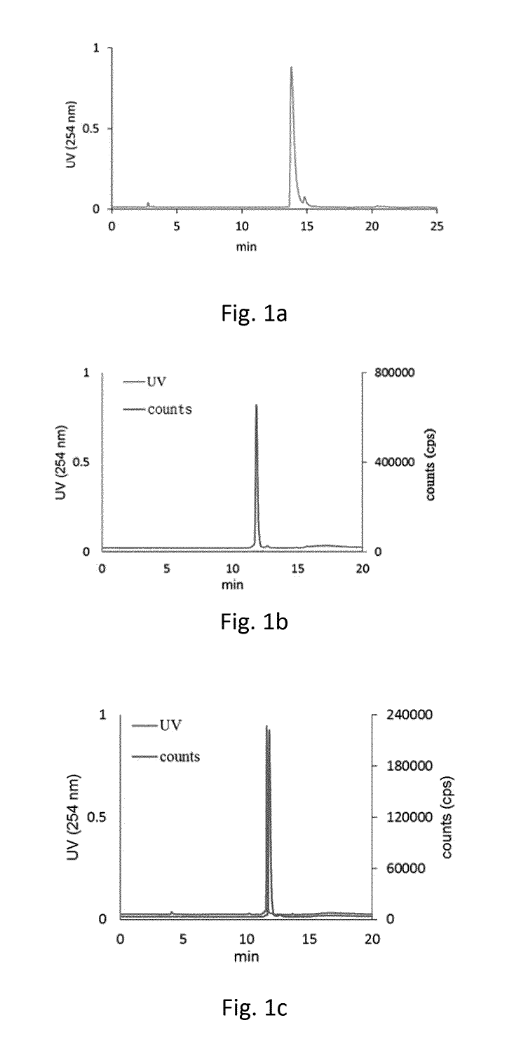

Nonradioactive FBuEA-GS 3 obtained from a parallel experiment could be resolved into two isomers in a ratio of 9:1 using analytic chiral HPLC (FIG. 1). For preparation purposes, a mixture of the two isomers of [¹⁸F]FBuEA-GS 3 obtained from semipreparative RP-HPLC purification was promptly used for all experiments, including radioligand enzymatic binding assays, cellular uptake study, ex vivo biodistribution experiments, and in vivo PET studies. From a series of experiments, [¹⁸F]FBuEA-GS 3 was obtained from [¹⁸F]F⁻ (end of bombardment, EOB), resulting in a radiochemical yield of 5%. Its specific activity and radiochemical purity were determined to be 33 GBq/μmol and 98% (FIG. 1b), respectively.

Bioassay of the competitive inhibitor FBuEA-GS 3 against the production of PGD2 To date, there is still no effective inhibitor of L-PGDS except AT-56 (IC50=95 mM), a dibenzocycloheptenyl tetrazolyl piperidine. This assay was performed via an indirect determination of the formation of PGD₂ in the presence of the competitive PGD2-acetylcholineesterase conjugate, which cleaves acetylthiocholine and the substrate 5,59-dithiobis(2-nitrobenzoic acid) to yield a colored 5-thio-2-nitrobenzoic acid with an absorbance of visible light at λₘₐₓ of 412 nm.

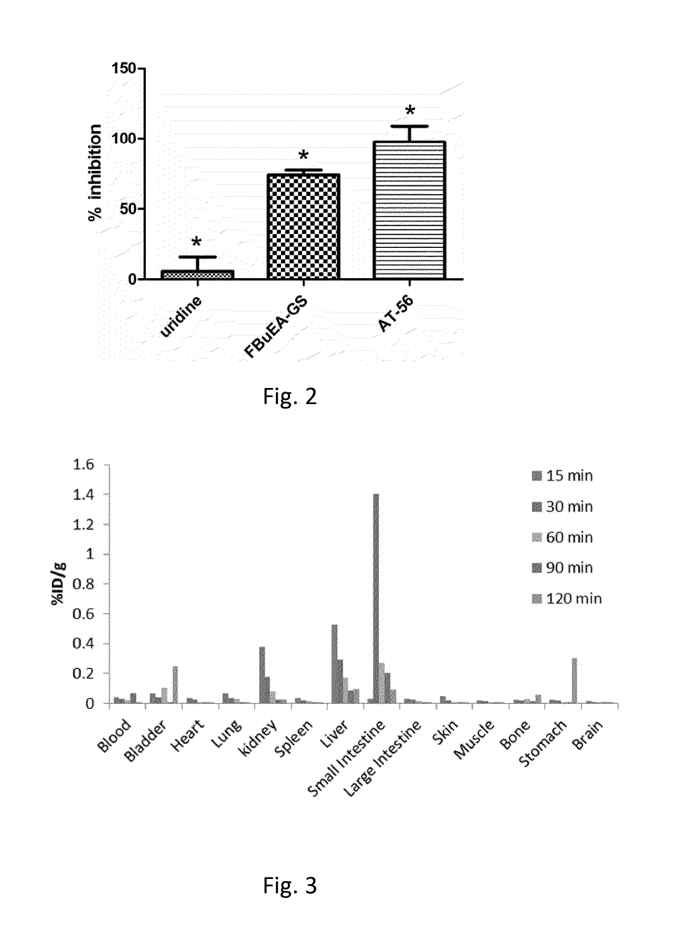

According to the IC₅₀ value of AT-56, working concentrations of 200 μM of substrates were required to ensure that AT-56 could be used as a positive control (FIG. 2). The relatively large deviation of uridine (5.6±14.3%) reflects the complexity of sequential assays. The observed inhibition was relatively higher than that observed in previous studies. Compared to the AT-56 positive control that, showed complete inhibition (97.6±16.0%), FBuEA-GS 3 (74.1±4.8%) data were significant.

Radioligand Enzymatic Binding Assays

L-PGDS catalyzes the oxidation of prostaglandin H2 (PGH₂), a metabolite (also known as a prostanoid) derived from arachidonic acid (AA) through oxidation and reduction via the catalysis of COX enzymes. For comparison, mPGES-1, the counterpart of L-PGDS that catalyzes the formation of PGE₂ from COX-derived PGH₂, was also used in this study. GST enzymes catalyze the conjugation of GSH to [¹⁸F]FBuEA 2 without having any significant binding to its metabolite [¹⁸F]FBuEA-GS 3. Thus, GST-P1 and GST-A1-1 enzymes were only used as negative controls. The specific activities of the enzymes are in the following order: mPGES-1 (2.2 units/mL)>L-PGDS of three species (2.4×10³ units/mL)>GST-P1≈GSTA1-1 (5×10⁻⁴ units/mL).

TABLE 1
Tabulated binding affinities of enzymes to [¹⁸F]FBuEA-
GS 3 according to one embodiment of the present invention

enzyme

| | COX-1 | COX-2 | m-PGES | GST-α |
|---|---|---|---|---|
| binding | 52 | 74.2 | negligible | negligible |
| ratio (%) | | | | |

| | enzyme | | | |
|---|---|---|---|---|
| | L-PGDS | L-PGDS | L-PGDS | |
| | (mouse | (rat | (human | |
| | recombinant) | recombinant) | recombinant) | GST-π |
| binding | 16.3 | 19.2 | 21.7 | negligible |
| ratio (%) | | | | |

Interestingly, mPGES-1 with a 1000-fold greater specific activity than that of L-PGDS did not show any binding affinity. The weak binding affinities of GSTA1-1 and GSTP1 could not be rationalized by lower specific activities because L-PGDS had similar specific activities (5-fold excess) and exhibited substantial binding.

Biological Testing

The accumulation of radioactivity of [¹⁸F]FBuEA-GS 3 was higher in tumor cells compared to that of normal cells (9% vs. 6%, not illustrated). Although the difference in tracer uptake between C6 glioma and fibroblast lies within the statistic error (p, 0.001 at 0 min and p.0.05 at rest time points), the accumulation level in C-6 glioma cell is higher. The accumulation pattern also differed from that of [¹⁸F]FbuEA 2, which had a lower uptake in tumor cells compared to normal cell (not illustrated). After reaching a steady state (approximately 15 min), the preferential radioactivity accumulation in tumors cell was maintained but then steadily decreased. The in vivo half-life (t_{1/2}) of [¹⁸F]FBuEA-GS3 was determined to be 60 min.

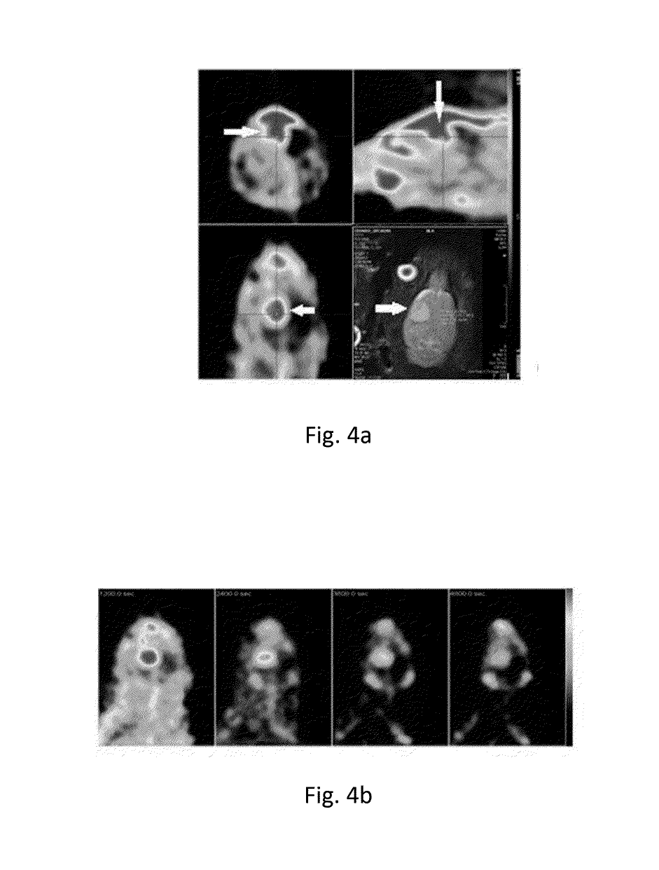

Based on the half-life of 1 h, the in vivo PET imaging test using micro PET were used in a 2-hr dynamic study, and the distribution of radioactivity in a rat was determined (FIG. 3). Fourteen different tissue samples were collected for the biodistribution study of 1.0-1.5 mCi of [¹⁸F]FBuEA-GS3 injected in a rat. The radioactivity was mainly localized in the excretory system. Only a limited amount of compound 3 was found in the brain (0.05% ID/g). Because of the quantitative features of PET, the radioactivity in tumor and normal tissues could be differentiated. [¹⁸F]FBuEA-GS3 was subsequently evaluated as a tracer for imaging a rat with a brain tumor (FIG. 4a). The tumor was successfully inoculated in the upper right part of the brain as confirmed by MRI imaging. The same rat was then taken for the measurement of gamma photons emitted from [¹⁸F]FBuEA-GS3, which was injected within 72 h after MRI imaging. The reconstructed images from all three cross sections showed a clear hot spot coinciding to the tumor region detected by MRI imaging. The dynamic PET images at coronal section from 0-120 min indicated that both the signal intensities on the tumor and regions other than tumor lesion decreased concomitantly. The imaging results were consistent with the results of the in vitro radioactivity accumulation study (FIG. 4b). The imaging experiments have been performed twice independently for two different C6-glioma rats using two PET scanner machines. Both the radioactivity accumulation levels in tumor lesions of the two rats are obvious but quantitative comparison has not been carried out.

While the invention can be subject to various modifications and alternative forms, a specific example thereof has been shown in the drawings and is herein described in detail. It should be understood, however, that the invention is not to be limited to the particular form disclosed, but on the contrary, the invention is to cover all modifications, equivalents, and alternatives falling within the spirit and scope of the appended claims.

What is claimed is:

1. A PET (positron emission tomography) tracer for imaging a tumor or a neurological disorder that overexpresses L-PGDS (Lipocalin-type prostaglandin D synthase) enzyme, comprising following formula or pharmaceutical salts thereof.

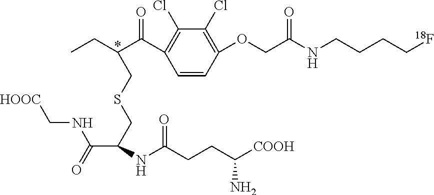 [SMILES: CCC(CSC[C@@H](NC(=O)CC[C@@H](N)C(=O)O)C(=O)NCC(=O)O)C(=O)c1ccc(OCC(=O)NCCCC[18F])c(Cl)c1Cl]

2. The PET tracer as claimed in claim 1, wherein the tumor is a brain tumor.

3. The PET tracer as claimed in claim 1, wherein the PET tracer is chiral.

4. A method for imaging a tumor or a neurological disorder that overexpresses L-PGDS (Lipocalin-type prostaglandin D synthase) enzyme, comprising:
administering an effective amount of a PET (positron emission tomography) tracer comprising following formula or pharmaceutical salts thereof to a subject; and 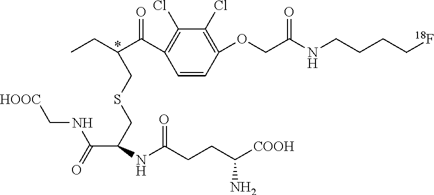 [SMILES: CCC(CSC[C@@H](NC(=O)CC[C@@H](N)C(=O)O)C(=O)NCC(=O)O)C(=O)c1ccc(OCC(=O)NCCCC[18F])c(Cl)c1Cl]
performing a PET imaging to the subject so as to imaging the tumor or the neurological disorder that overexpresses L-PGDS enzyme.

5. The method for imaging a tumor or a neurological disorder that overexpresses L-PGDS enzyme as claimed in claim 4, wherein the tumor is a brain tumor.

6. The method for imaging a tumor or a neurological disorder that overexpresses L-PGDS enzyme as claimed in claim 4, wherein the PET tracer is chiral.

7. The method for imaging a tumor or a neurological disorder that overexpresses L-PGDS enzyme as claimed in claim 4, wherein the neurological disorder is Alzheimer's disease.
